(19) United States
(12) Patent Application Publication
(10) Pub. No.: US 2026/0263187 A1
(43) Pub. Date: Sep. 10, 2026

(54) INSERTION TOOL WITH LIGHT SOURCE

(71) Applicants: NATIONAL UNIVERSITY CORPORATION KOCHI UNIVERSITY, Kochi-shi, Kochi (JP); NIREC CORPORATION, Kochi-shi, Kochi (JP)

(72) Inventors: Takayuki SATO, Kochi-shi (JP); Tettsuo SUMIDA, Kochi-shi (JP)

(73) Assignees: NATIONAL UNIVERSITY CORPORATION KOCHI UNIVERSITY, Kochi-shi, Kochi (JP); NIREC CORPORATION, Kochi-shi, Kochi (JP)

(21) Appl. No.: 19/153,833

(22) PCT Filed: Feb. 9, 2023
(86) PCT No.: PCT/JP2023/004471

§ 371 Date: Aug. 5, 2025

(51) Int. Cl. A61B 90/30; A61B 1/00; A61B 1/313; A61B 90/00

(52) U.S. Cl. CPC A61B 90/30; A61B 1/00131; A61B 1/3132; A61B 90/39; A61B 2090/309; A61B 2090/3937

(57) ABSTRACT

An insertion tool that is inserted into a natural opening of the body, such as the vagina or rectum, and when inserted into the body, causes light from a distal end of the insertion tool to efficiently and safely enter a tissue inside a body cavity, and allows the light that has entered the tissue inside the body cavity from the distal end to be clearly observed from an abdominal cavity side. The insertion tool, which is inserted into a natural opening of the body, includes a light source at a proximal end of a cylindrical body formed of a light-conducting resin, and a light-projecting portion at a distal end. The light-projecting portion at the distal end has a structure configured to project light, which has entered the proximal end, in a radially outward direction of the cylindrical body at the distal end with a high intensity.

### TECHNICAL FIELD

The present invention relates to an insertion tool with a light source, for use in a laparoscopic total hysterectomy surgery and the like.

### BACKGROUND ART

Generally, a laparoscopic total hysterectomy surgery involves cutting the round ligament, infundibulopelvic ligament, uterosacral ligament, uterine artery, ovarian artery, and the like, and detaching the bladder. The end of the uterus extends into the upper part of the vagina and is surrounded by the vaginal fornix (a wing-like extension at the upper end of the vagina). The uterus is dissected from the vaginal canal at or near the vaginal fornix using a scalpel inserted into the abdominal cavity under laparoscopic observation and is removed from the body, and the vaginal incision is closed with sutures. The line along which the uterus is dissected is called the “dissection line”. It is important to determine a dissection line with which the total hysterectomy can be performed with minimal invasiveness. However, it is difficult to determine such a dissection line because the vaginal fornix is not directly visible when observed from the abdominal cavity.

In a conventional method for determining the dissection line, a surgeon transvaginally inserts his/her finger, or a rigid pipe as described in Patent literature 1 or the like (examples of a commercially available product thereof include Vagi-pipe manufactured by Hakko Co., Ltd.) into the body and presses it against the vaginal fornix (the wing-like extension at the upper end of the vagina surrounding the end of the uterus). While manipulating and pressing the finger or the rigid pipe, the movement of the vaginal fornix is observed with the laparoscope in the abdominal cavity, and the dissection line is determined from the observed movement. However, this method only allows for a rough estimate of the actual position of the dissection line.

Another method for determining the dissection line is to observe the boundary between the uterus and the vagina with a laparoscope by transilluminating the boundary from the vaginal side. Examples thereof include: a method in which an optical fiber connected to a light source outside the body is passed through a tube and transvaginally inserted into the body, the end of the optical fiber at the tip of the tube is moved along the vaginal fornix, and light that has passed through the vaginal fornix is observed with a laparoscope in the abdominal cavity to determine the dissection line (Patent literature 2); a method in which an optical fiber is embedded in the wall of a pipe to be inserted into the vagina, light from an external light source outside the body is made enter the optical fiber and projected from the end of the optical fiber at the tip of the pipe, and the light that has passed through the vaginal fornix is observed with a laparoscope in the abdominal cavity to determine the dissection line (Patent literature 3); a method in which a resin ring with an embedded light-emitting diode (LED) is attached to the distal end of a pipe to be inserted into the vagina, the power is supplied to the LED through a wire from outside of the body to cause the LED to emit light, and the light that has passed through the vaginal fornix is observed with a laparoscope in the abdominal cavity to determine the dissection line (Patent literature 4) ; and a method in which LEDs are arranged in a ring shape on the rim of a cervical cup that is transvaginally inserted into the body and pressed against the vaginal fornix, and light emitted by the LEDs is observed with a laparoscope in the abdominal cavity to determine the dissection line (Patent literature 5).

However, when the light emitted from the light source outside the body is passed through the optical fiber to illuminate the boundary between the uterus and the vagina from the vaginal side as described in Patent literatures 2 and 3, the transmission loss of light becomes significant, making it difficult to determine the dissection line. Furthermore, the optical fiber that connects the light source outside the body and the device inside the vagina often interferes with the surgical procedure.

On the other hand, placing the LEDs inside the body as described in Patent literatures 4 and 5 increases the risks associated with the medical equipment, including the risk of electric shock and the risk of a temperature rise of the internal device.

### CITATION LIST

### Patent Literature

PTL1: Japanese Patent Application Laid-Open No. 2004-41395

PTL 2: Japanese Patent No. 4038590

PTL 3: Japanese Patent Application Laid-Open No. Hei. 11-336

PTL 4: Japanese Patent No. 6133423

PTL 5: Japanese Patent Application Laid-Open No. 2017-202317

### SUMMARY OF INVENTION

### Technical Problem

Regarding the above-mentioned prior art, an object of the present invention is to provide an insertion tool that is inserted into a natural opening of the body, such as the vagina or rectum, the insertion tool, when inserted into the body, causing light from a distal end of the insertion tool to efficiently and safely enter a tissue inside a body cavity, and allowing the light that has entered the tissue inside the body cavity from the distal end to be clearly observed from an abdominal cavity side.

### Solution to Problem

The present inventor has found that (i) when an insertion tool to be inserted into a natural opening of the body is a cylindrical body formed of a light-conducting resin, a light source is disposed directly at a proximal end of the insertion tool, and a distal end of the insertion tool serves as a light-projecting end, light emitted from the light source can be efficiently sent to the distal end, (ii) when the distal end is formed into a specific shape, the distal end of the cylindrical body inserted into the natural opening comes into close contact with a tissue inside a body cavity, thereby making the light projected from the distal end efficiently enter the tissue inside the body cavity, and further, (iii) when the distal end is formed into a specific shape, the intensity of light that is projected from the distal end and travels in a radially outward direction of the cylindrical body is high, making it easier to observe the light emission of the tissue inside the body cavity from a serosal membrane side of the tissue inside the body cavity, thereby completing the present invention.

That is, the present invention provides an insertion tool with a light source, which is inserted into a natural opening of the body, the insertion tool including the light source at a proximal end of a cylindrical body formed of a light-conducting resin, and a light-projecting portion at a distal end, the light-projecting portion having a structure configured to project light, which has entered the proximal end, in a radially outward direction of the cylindrical body at the distal end with a high intensity.

### Advantageous Effects of Invention

According to the present invention, the cylindrical body to be inserted into the natural opening of the body such as the vagina or rectum is formed of the light-conducting resin, and the cylindrical body includes the light source at its proximal end and the light-projecting portion at its distal end. Thus, the light emitted from the light source enters the proximal end and projects from an annular convex portion at the distal end without the transmission loss that would be caused by an optical fiber.

Furthermore, the light-projecting portion at the distal end has a structure configured to project the light, which has entered the proximal end, in the radially outward direction of the cylindrical body with a high intensity. Thus, when the insertion tool of the present invention is inserted into the natural opening of the body such as the vagina or rectum with the light source at the proximal end being turned on, the light projected from the light-projecting portion can be clearly observed from the serosal membrane side of the tissue inside the body cavity at the insertion site, making it easy to determine the dissection line from the serosal membrane side.

In addition, in this insertion tool, the light source is provided at the proximal end of the cylindrical body, so that the light source is located outside the body. Therefore, the risk of electric shock and the risk of a temperature rise, which are substantial if the light source is disposed inside the body, are avoided.

### BRIEF DESCRIPTION OF DRAWINGS

FIG. 1A is a perspective view of an insertion tool 1A with a light source of an example with a cap opened, as viewed from a distal end side.

FIG. 1B is a perspective view of the insertion tool 1A with a light source of the example with the cap closed, as viewed from a proximal end side.

FIG. 1C is a side view of the insertion tool 1A with a light source of the example with the cap opened.

FIG. 1D includes a sectional view of the insertion tool 1A with a light source of the example with the cap opened, and views as seen from arrow a and from arrow b.

FIG. 2 is a vertical sectional view of an annular convex portion 20A, showing an action of the annular convex portion 20A formed as a light-projecting portion of the insertion tool 1A with a light source of the example at the distal end.

FIG. 3A is a perspective view of a typical vagina into which the insertion tool 1A with a light source of the example has been inserted, as viewed from an abdominal cavity side.

FIG. 3B is an explanatory diagram of a position of the dissection line of the uterus determined in the typical vagina into which the insertion tool 1A with a light source of the example has been inserted.

FIG. 4A is a perspective view of the deep vagina into which the insertion tool 1A with a light source of the example has been inserted, as viewed from the abdominal cavity side.

FIG. 4B is an explanatory diagram of a position of the dissection line of the uterus determined in the deep vagina into which the insertion tool 1A with a light source of the example has been inserted.

FIG. 5 is a vertical sectional view of an annular convex portion, showing an action of the annular convex portion formed as a light-projecting portion 20B of an insertion tool with a light source of an example at the distal end.

FIG. 6 is a vertical sectional view of an annular convex portion, showing an action of the annular convex portion formed as a light-projecting portion 20C of an insertion tool with a light source of an example at the distal end.

FIG. 7 is a vertical sectional view of an annular convex portion, showing an action of the annular convex portion formed as a light-projecting portion 20D of an insertion tool with a light source of an example at the distal end.

FIG. 8 is a vertical sectional view of a blasting processed portion, showing an action of the blasting processed portion formed as a light-projecting portion 20p of an insertion tool with a light source of an example at the distal end.

FIG. 9 is a vertical sectional view of a light diffusing agent-containing layer, showing an action of the light diffusing agent-containing layer formed as a light-projecting portion 20q of an insertion tool with a light source of an example at the distal end.

FIG. 10 is a vertical sectional view of a light-projecting portion 20Apq showing an action of a portion that is the annular convex portion formed as the light-projecting portion 20Apq of an insertion tool with a light source of an example at the distal end, the annular convex portion having a blasting processed portion and containing a light diffusing agent.

FIG. 11 is a vertical sectional view showing an action of an insertion tool with a light source of a comparative example at the distal end.

FIG. 12 is a vertical sectional view showing an action of an insertion tool with a light source of a Comparative example at the distal end.

FIG. 13A is a side view of an insertion tool 1B of an example.

FIG. 13B is a sectional view of the insertion tool 1B of the example.

FIG. 14 is a photograph showing a light emission test of the insertion tool 1A of the example covered with a skin sheet.

FIG. 15A is a photograph showing a light emission test of an insertion tool with tip R processing.

FIG. 15B is a photograph showing a light emission test of an insertion tool without tip R processing.

### DESCRIPTION OF EMBODIMENTS

An insertion tool with a light source (hereinafter also simply referred to as “insertion tool”) of the present invention will be described in detail below with reference to the drawings by way of an example of a vaginal-use insertion tool that is used in a laparoscopic total hysterectomy surgery and the like. In the respective drawings, the same reference numerals represent the same or equivalent constituent elements.

Note that the insertion tool of the present invention is not limited to vaginal use and may also be configured as an insertion tool to be inserted into a natural opening of the body such as the rectum.

### Overall Structure

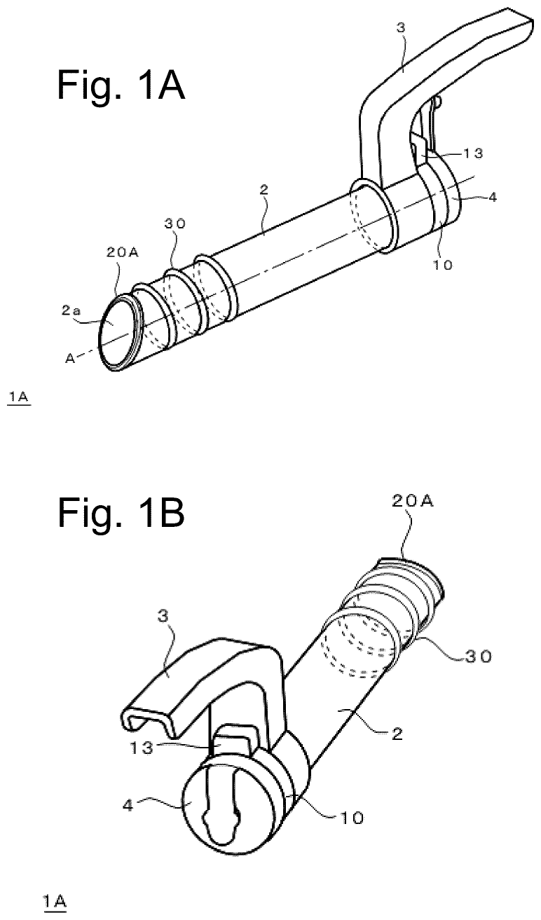

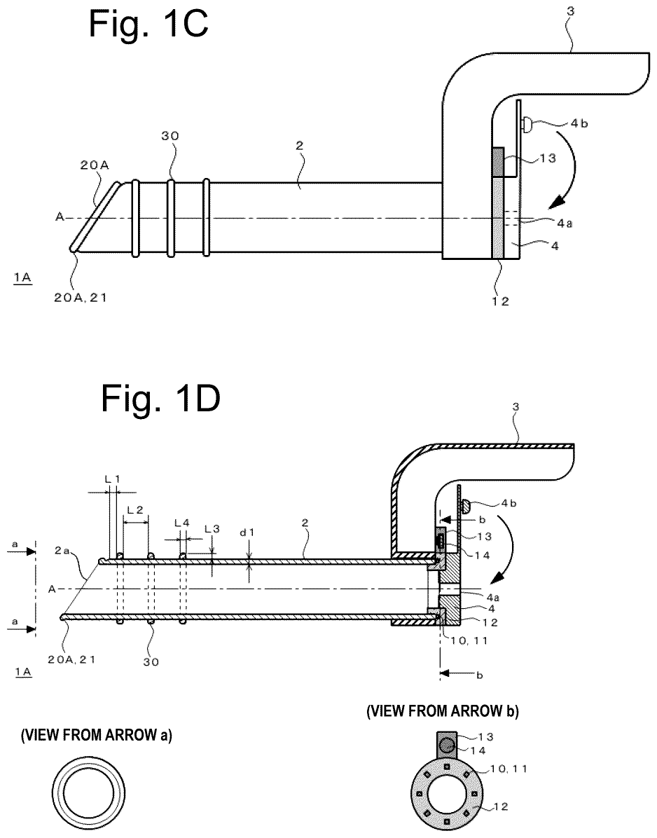

As illustrated in FIG. 1A, FIG. 1B, FIG. 1C, and FIG. 1D, a vaginal-use insertion tool 1A with a light source as one example of the present invention includes a cylindrical body 2 that has a substantially cylindrical shape and is to be inserted into the vagina. The length of the cylindrical body 2 in an axis A direction is determined such that a proximal end thereof is outside the body when the cylindrical body 2 is inserted into the vagina. The length is, for example, 180 mm or more. Furthermore, a wall thickness d1 of the cylindrical body 2 is preferably about 2 to 4 mm.

The distal end of the cylindrical body 2 has an opening, and an opening surface 2a of the cylindrical body 2 is inclined with respect to the axis A of the cylindrical body 2 to account for the fact that the posterior vaginal fornix is located deeper than the anterior vaginal fornix.

A handle 3 is attached near the proximal end of the cylindrical body 2 as necessary, and a cap 4 is fitted on the cylindrical body 2. An opening 4a is formed in the center of the cap 4 for inserting a tool used during surgery, and a plug 4b that fits into the opening 4a as shown by the arrow is formed integrally with the cap 4. The handle 3 and the cap 4 can be configured in the same manner as that for the insertion tool described in Patent Literature 1.

On the other hand, the insertion tool of the present invention is mainly configured so that a light source is attached to the proximal end of the cylindrical body 2 and the cylindrical body 2 is formed of a light-conducting resin. This configuration reduces the transmission loss of light when the light that has been emitted from the light source and has entered the proximal end is guided to the distal end.

Examples of the light-conducting resin that can be used include polymethyl methacrylate, polycarbonate, and polystyrene.

As the light source, for the insertion tool 1A of the present example, a ring-shaped lighting device 10 in which a plurality of chip LEDs 11 are embedded is provided. The ring-shaped lighting device 10 directs the light-emitting surfaces of the chip LEDs 11 toward the distal end of the cylindrical body 2.

Furthermore, the insertion tool of the present invention is also mainly configured so that it has a light-projecting portion provided at the distal end of the cylindrical body 2, and that the light-projecting portion has a structure configured to project light, which has entered the proximal end, with a high intensity in a radially outward direction at the distal end of the cylindrical body, i.e., the intensity of light projected in a radially outward direction of the cylindrical body is high in the intensity distribution of the light projected from the distal end.

The structure of the light-projecting portion at the distal end can have various configurations as long as the light that has entered the proximal end is projected in a radially outward direction at the distal end with a high intensity. For example, the insertion tool 1A with a light source of the present example includes an annular convex portion that is convex in the radially outward direction of the cylindrical body 2, the annular convex portion serving as a light-projecting portion 20A at the distal end. This light-projecting portion (annular convex portion) 20A includes a curved surface 21 with which a tissue inside a body cavity comes into close contact when the insertion tool 1A is inserted into the body cavity. Since the curved surface 21 is convex in the radially outward direction of the cylindrical body 2, the light emitted from the light source 10 can be projected from the light-projecting portion (annular convex portion) 20A in the radially outward direction of the cylindrical body 2 with a high intensity. In addition, when the insertion tool 1A is inserted into the vagina, the tissue inside the body cavity is in close contact with the curved surface 21, so that the light projected from the curved surface 21 enters the tissue inside the body cavity and passes therethrough without any loss of light intensity in a gap between the curved surface 21 and the tissue inside the body cavity. This effect makes it possible to clearly observe the light-projecting portion inside the body cavity using a laparoscope inserted into the body cavity on the serosal membrane side of the tissue inside the body cavity.

### Light Source

In the present invention, the light source 10 is attached to the proximal end of the cylindrical body 2 without the use of an optical fiber. An LED or the like is preferably used as the light source. The light source 10 is preferably used so that the light-emitting surface thereof faces the end face of the proximal end of the cylindrical body 2. More specifically, for example, as illustrated in FIG. 1D, a plurality of chip LED 11 are arranged in a ring-like shape along the end face of the proximal end of the cylindrical body 2 with the light-emitting surfaces thereof facing the end face. In this case, the chip LEDs 11 may be embedded in a ring-shaped member 12 formed of a resin having a refractive index similar to that of the cylindrical body 2, the light-emitting surfaces of the chip LEDs 11 may be directed toward the end face of the proximal end of the cylindrical body 2, and the ring-shaped member 12 and the end face of the proximal end of the cylindrical body 2 may be bonded to each other using an adhesive having a refractive index similar to that of the cylindrical body 2. Alternatively, the chip LEDs 11 may be directly bonded to the end face of the proximal end of the cylindrical body 2 using an adhesive having a refractive index similar to that of the resin constituting the cylindrical body 2.

An emission wavelength of the light source 10 is preferably in a range of visible light to near infrared light (about 400 to 1000 nm) from the viewpoint of visibility of the light-projecting site when observed through the laparoscope.

Furthermore, it is preferable to provide a battery box 13, which houses a battery 14 that serves as the power source for the chip LEDs 11, at the proximal end of the cylindrical body 2. In the insertion tool 1A of the example, the battery box 13 is provided in parallel with the ring-shaped member 12. This configuration prevents wiring of the light source 10 from interfering with the surgical procedure.

### Annular Convex Portion

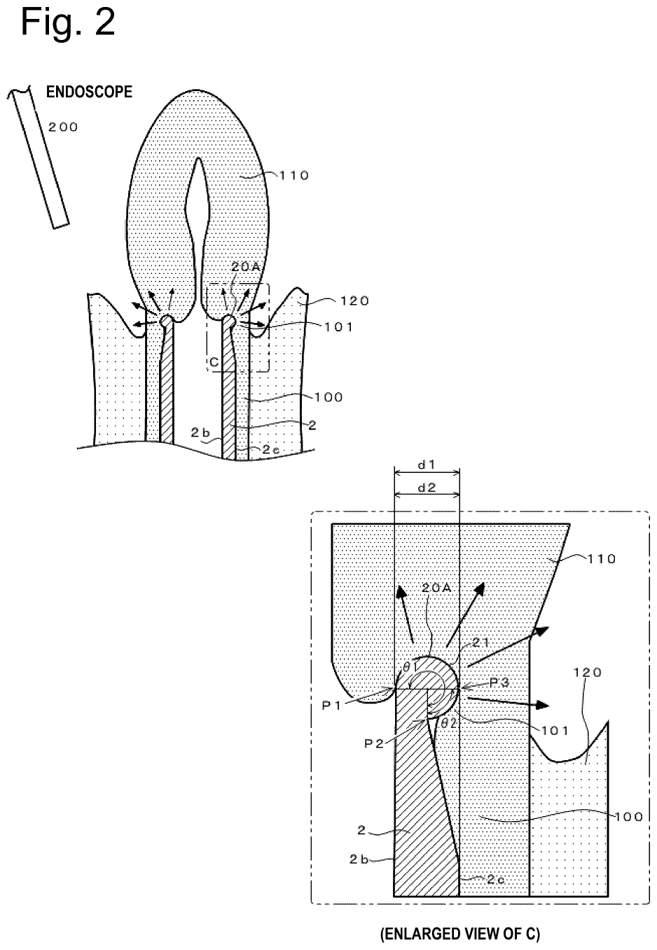

FIG. 2 is a vertical sectional view (sectional view along the axis A of the cylindrical body) of the annular convex portion 20A in a state where the insertion tool 1A of the example is inserted into a vagina 100 and the annular convex portion 20A at the distal end of the insertion tool 1A is pressed against a vaginal fornix 101. This annular convex portion 20A includes the curved surface 21 that is convex in the radially outward direction of the cylindrical body 2. As illustrated in the drawing, the curved surface 21 is not formed in a radially inward direction of the cylindrical body 2 beyond an inner end point P1 of an arc that forms the curved surface 21, but it protrudes in the radially outward direction of the cylindrical body 2 beyond an outer end point P2 of the arc that forms the curved surface 21. Furthermore, on the curved surface 21, the inner end point P1, a point (outer protruding point) P3 that protrudes most in the radially outward direction, and the outer end point P2 smoothly continue, allowing the curved surface 21 to be shaped to be in close contact with a tissue 120 inside a body cavity both on the inner end point P1 side of the outer protruding point P3 and on the outer end point P2 side of the outer protruding point P3.

More specifically, in the sectional view illustrated in FIG. 2, the inner end point P1 of the arc of the curved surface 21 is on an inner surface 2b of the cylindrical body 2, and the curved surface 21 is located outside the inner end point P1 in the radial direction of the cylindrical body 2. Furthermore, the curved surface 21 is an arc of about ¾ circle (θ1:240 to 285°) formed within the wall thickness of the cylindrical body 2. The curved surface 21 continues for about ¼ circle (θ2:60 to 105°) to the proximal end side from the outer protruding point P3 that protrudes most in the radially outward direction. Thus, when the cylindrical body 2 is pressed against the vaginal fornix 101, almost the entire surface of the curved surface 21 comes into close contact with the tissue inside the body cavity.

Note that the entire surface of the curved surface 21 does not necessarily need to come into close contact with the tissue inside the body cavity. However, it is preferable that the curved surface 21 come into close contact with the tissue inside the body cavity on the outer protruding point P3 and its inner end point P1 side as well as its outer end point P2 side, preferably on a protruding region having the outer protruding point P3 positioned in the center.

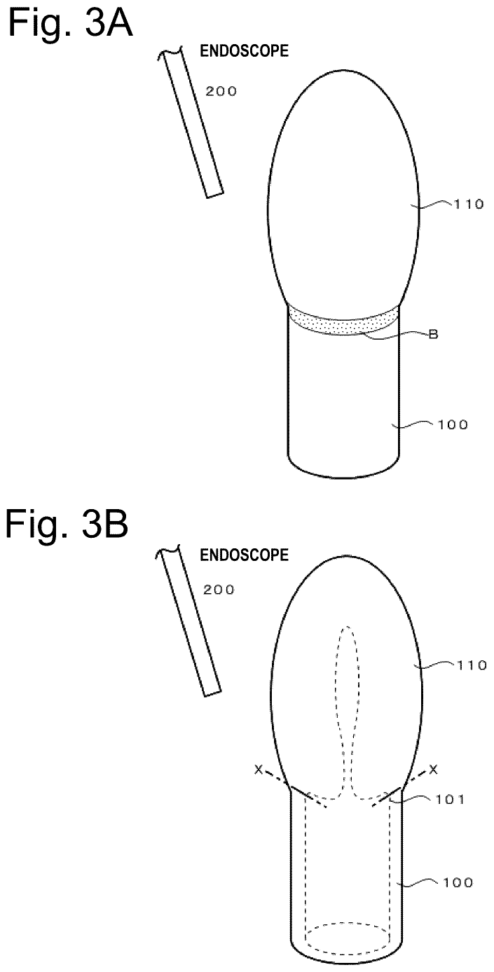

When the insertion tool 1A including the annular convex portion 20A at the distal end illustrated in FIG. 2 is transvaginally pressed against the vaginal fornix 101, the light source 10 at the proximal end is turned on, and light is projected from the annular convex portion 20A at the distal end, the light diffuses upward (in the axis A direction) and to the sideway as shown by the arrows in the drawing. The light diffuses especially to the oblique sideway with high intensity. Thus, as illustrated in FIG. 3A, the light projected from the annular convex portion 20A pressed against the vaginal fornix 101 can be observed from the abdominal cavity side as a light-projecting portion B with an endoscope 200, making it possible to appropriately determine a dissection line X as illustrated in FIG. 3B.

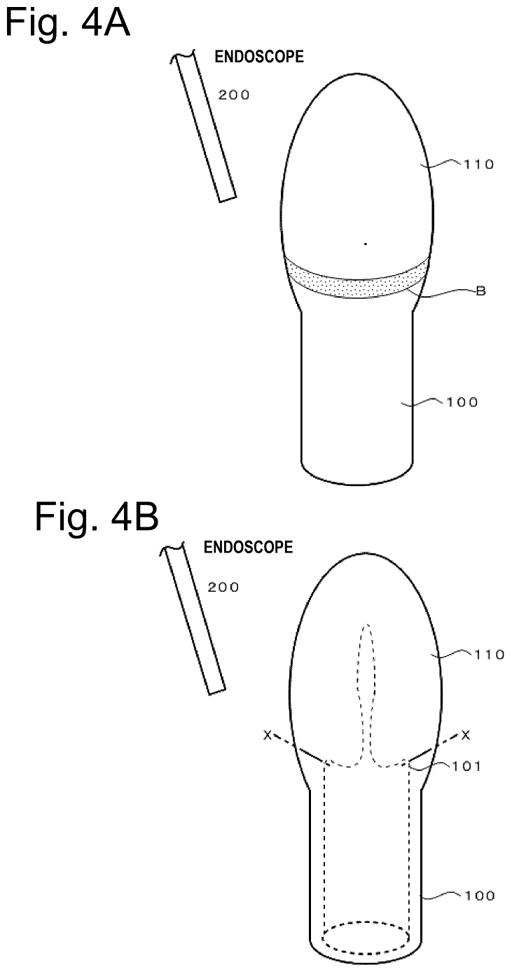

The shapes of the vagina 100 and uterus 110 as viewed from the abdominal cavity may be the same as those illustrated in FIG. 3A and FIG. 3B, or the vagina 100 may be long and the vaginal fornix 101 may be recessed as illustrated in FIG. 4A and FIG. 4B. However, even in the latter case, when the insertion tool 1A of the example is used, it is possible to observe the light-projecting portion B corresponding the position of the vaginal fornix 101. Thus, as illustrated in FIG. 4B, it is possible to appropriately determine the dissection line X.

In contrast, when a distal end 2x of the cylindrical body 2 forms a flat surface perpendicular to the axis A direction, as in the insertion tool of a comparative example illustrated in FIG. 11, the emission intensity, at the distal end 2x, of the light, which has been emitted from the light source and has been caused to enter the proximal end of the cylindrical body 2, is high in the axis A direction, but the intensity of the light projected to the sideway is low. Furthermore, as illustrated in an enlarged view in the same drawing, the cylindrical body 2 does not come into close contact with the tissue 120 inside the body cavity in a region 2r extending from the corner of the distal end 2x toward the proximal end. The refractive index of the cylindrical body 2 (e.g., 1.49 in a case of acrylic resin), the refractive index of the tissue inside the body cavity (the refractive index of a biological tissue is about 1.55, and the refractive index of water contained therein is 1.33), and the refractive index of a gap between them (the refractive index of air is 1.00) are different from each other, causing reflection to occur at the interface between them, and the intensity of the light passing through the tissue inside the body cavity from the distal end 2x through the sideway of the cylindrical body 2 becomes even lower. For this reason, it becomes difficult to observe the light projected from the distal end 2x of the cylindrical body 2 as the light-projecting portion from the abdominal cavity side, making it difficult to determine the dissection line X.

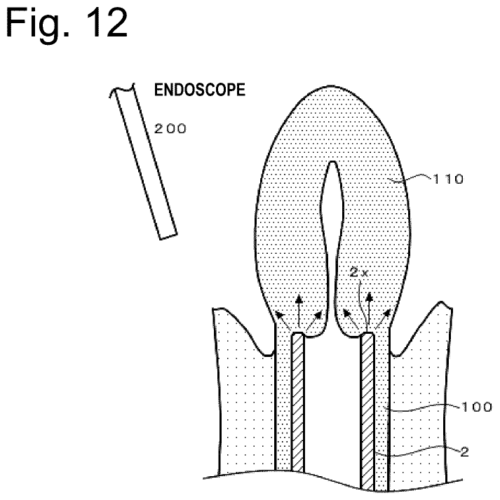

Furthermore, when the corners of the distal end 2x of the cylindrical body 2 are simply rounded as in the insertion tool of the comparative example illustrated in FIG. 12, the diffusion of the light projected from the distal end 2x becomes stronger than in the case illustrated in FIG. 11, but the intensity of the light projected to the sideway of the cylindrical body 2 is low. For this reason, by simply rounding the corners of the distal end 2x of the cylindrical body 2, it is still difficult to observe the light projected from the distal end 2x as the light-projecting portion from the abdominal cavity side.

### Modification of Light-Projecting Portion at Distal End

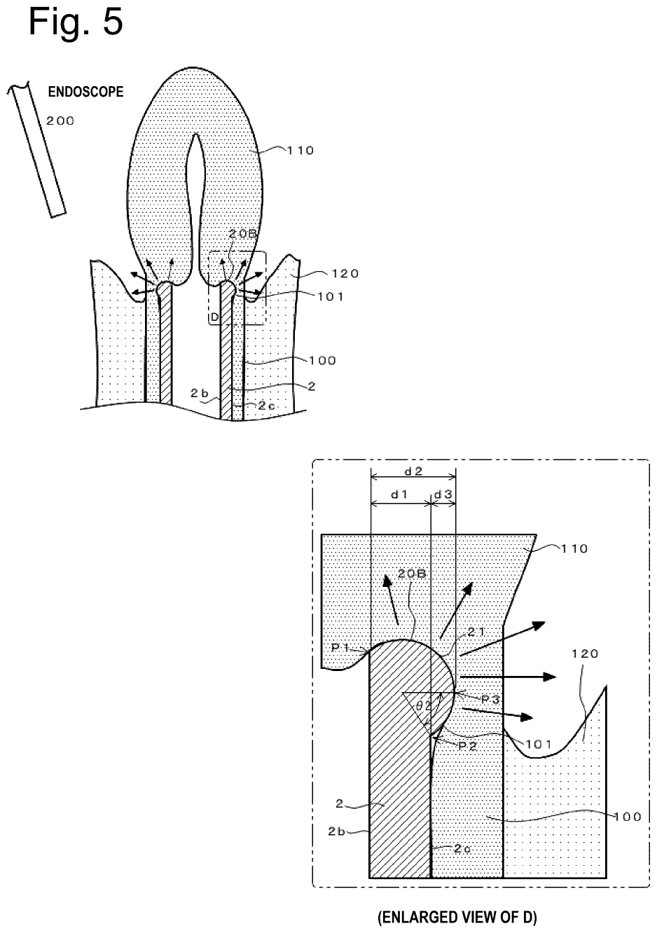

As illustrated in FIG. 3A or FIG. 3B, for clearly observing the light projected from the annular convex portion provided as the light-projecting portion at the distal end of the cylindrical body 2 from the abdominal cavity side, in the present invention, as in an annular convex portion 20B at the distal end of the cylindrical body 2 illustrated in FIG. 5, a radial thickness d2 of the annular convex portion 20B at the outer protruding point P3 may be made larger than the wall thickness dl of the cylindrical body 2. It is preferable that a difference d3 between the thickness d2 of the annular convex portion 20B and the wall thickness d1 of the cylindrical body 2 be ¼ or more of the wall thickness d1.

Note that, in the annular convex portion 20B of the insertion tool illustrated in FIG. 5, for improving the close contact between the annular convex portion 20B and the tissue inside the body cavity, the annular convex portion 20B has a smoothly continuous curved surface from the inner end point P1 through the outer protruding point P3 to the outer end point P2. Furthermore, the curved surface 21 continues for about ⅙ circle (θ2:40 to 70°) to the proximal end side from the outer protruding point P3.

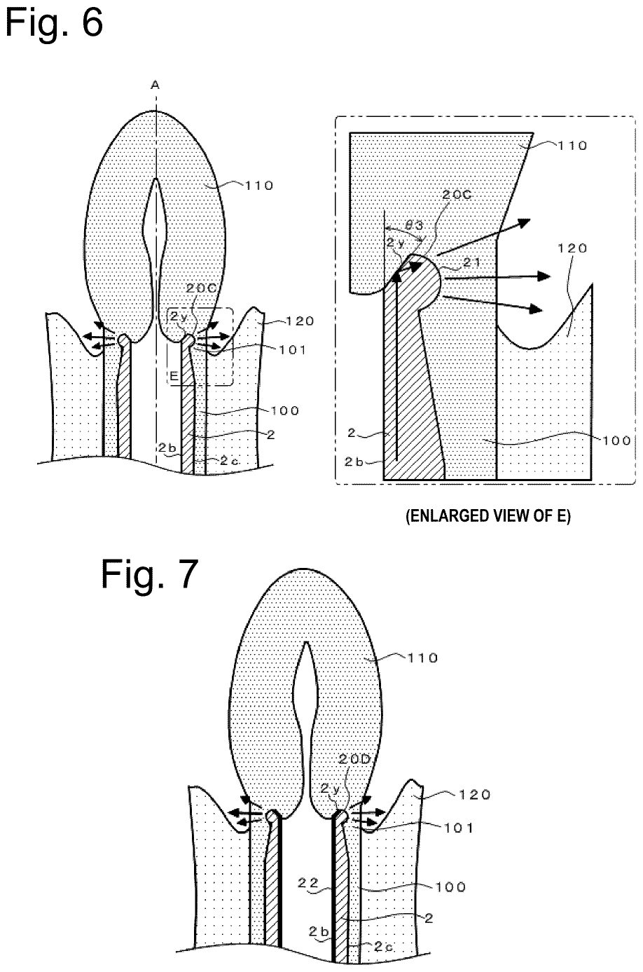

In contrast to the annular convex portion 20A illustrated in FIG. 2, an annular convex portion 20C at the distal end of the cylindrical body 2 illustrated in FIG. 6 is obtained by inclining the tip portion of the inner surface 2b of the cylindrical body 2 so that the opening diameter of the inner surface 2b of the cylindrical body 2 becomes wider as it approaches the tip of the cylindrical body 2. An inclination angle θ3 is preferably 35 to 60° from the axis A direction. By forming such an inclined surface 2y, the light that has been caused to enter the proximal end of the cylindrical body 2 can be reflected at the inclined surface 2y in the radially outward direction of the cylindrical body 2, and then projected from the curved surface 21 in the radially outward direction of the cylindrical body 2. Thus, the intensity of the light projected in the radially outward direction becomes higher. As a result, when the light is projected from the annular convex portion 20C illustrated in FIG. 6 to the tissue inside the body cavity, the light-projecting portion of the tissue inside the body cavity can be clearly observed from the abdominal cavity side.

The annular convex portion 20D at the distal end of the cylindrical body 2 illustrated in FIG. 7 is configured by forming a reflective film 22 on the inner surface 2b of the cylindrical body in the annular convex portion 20C illustrated in FIG. 6. The reflective film 22 may be formed of a metal film such as an aluminum vapor-deposited film, or may be formed using a commercially available mirror-effect spray paint. The presence of the reflective film 22 increases the intensity of the light reflected in the radially outward direction on the inclined surface 2y. Therefore, the annular convex portion 20D can be more clearly observed as the light-projecting portion from the abdominal cavity side.

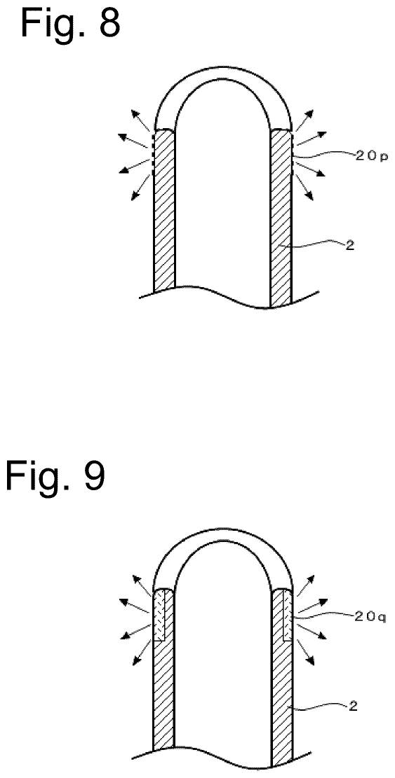

The light-projecting portion 20p of the cylindrical body 2 illustrated in FIG. 8 is obtained by rounding the corners at the distal end of the cylindrical body 2 and roughening the outer peripheral surface at the distal end. The roughened surface is preferably formed by blasting process.

Blasting process is a surface roughening technique for forming an infinite number of fine scratches on a surface, and can be performed using a laser processing machine, a sand blasting machine, or the like.

When the outer peripheral surface at the distal end of the cylindrical body 2 is roughened to become a roughened surface 20p, the light that has been caused to enter the proximal end of the cylindrical body 2 diffuses from the roughened surface 20p. Therefore, even when the roughened surface 20p is formed at the distal end of the cylindrical body 2, the light emission at the roughened surface 20p can be observed from the abdominal cavity side.

The light-projecting portion 20q of the cylindrical body 2 illustrated in FIG. 9 is configured by rounding the corners at the distal end of the cylindrical body 2, and forming a light diffusing agent-containing layer on the outer peripheral surface at the distal end. As the light diffusing agent to be contained in the light-projecting portion 20q, a known light diffusing agent used in lighting apparatus can be used, and for example, micron-sized silicone particles, polystyrene particles, or the like can be used.

When the light diffusing agent-containing layer 20q is provided on the outer peripheral surface at the distal end of the cylindrical body 2, the light that has been caused to enter the proximal end of the cylindrical body 2 diffuses from the light diffusing agent-containing layer 20q. Therefore, since the light diffusing agent-containing layer 20q is formed on the outer peripheral surface at the distal end of the cylindrical body 2, the light emission from the light diffusing agent-containing layer 20q can be observed from the abdominal cavity side.

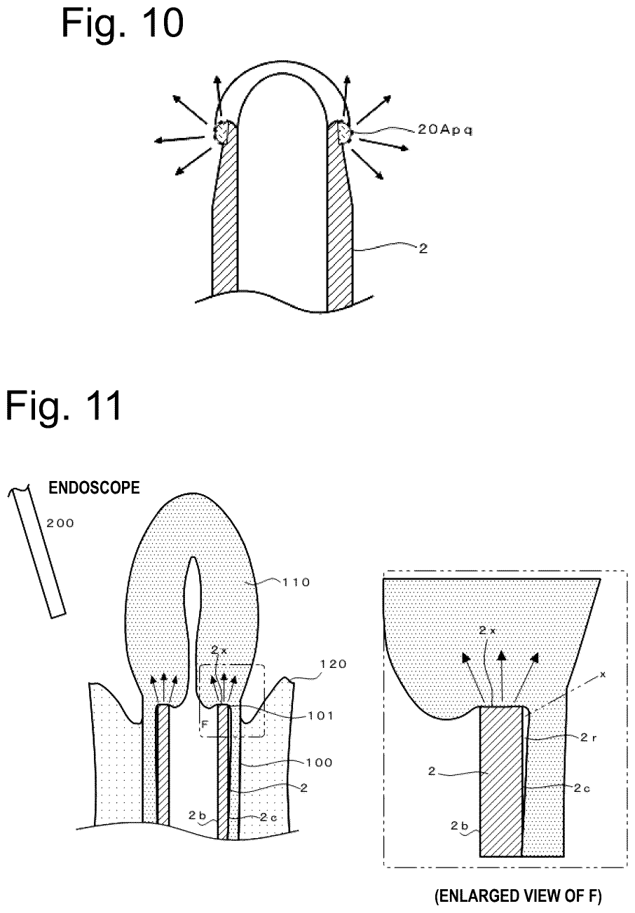

By combining the above-described configurations of the light-projecting portion (the annular convex portions 20A to 20D, the roughened surface 20p, and the light diffusing agent-containing layer 20q), the light that has entered the proximal end of the cylindrical body may be projected more strongly at the distal end in the radially outward direction of the cylindrical body. For example, the light-projecting portion 20Apq illustrated in FIG. 10 is obtained by forming a light diffusing agent-containing layer on the outer peripheral surface at the distal end of the cylindrical body 2, forming the distal end into the annular convex portion 20A illustrated in FIG. 2, and subjecting its curved surface to blasting process.

### Luminous Scale

The insertion tool with a light source of the present invention preferably includes a luminous scale on an outer peripheral surface at a predetermined distance from the light-projecting portion at the distal end of the cylindrical body, the luminous scale configured to emit the light, which has entered the proximal end, in a scale mark-like pattern. For example, as in the insertion tool 1A of the example illustrated in FIG. 1C and FIG. 1D, an annular convex portion 30 can be provided as a luminous scale around the entire periphery of the cylindrical body at a predetermined distance L1 from the light-projecting portion (annular convex portion) 20A at the distal end of the cylindrical body 2. A plurality of the luminous scales (annular convex portions) 30 are formed, and the luminous scales 30 are spaced apart by a predetermined interval L2.

In the present example, each luminous scale 30 is formed in a plane perpendicular to the axis A of the cylindrical body 2. Furthermore, each luminous scale 30 protrudes in a semi-cylindrical shape in a vertical section (section cut in the axial direction) of the cylindrical body 2. A protruding length L3 of the luminous scale 30 from an outer surface 2c of the cylindrical body is preferably ¼ times or more and 1 time or less the wall thickness d1 of the cylindrical body 2. Furthermore, a width L4 of the luminous scale in the axis A direction is preferably ¼ times or more and 1 time or less the wall thickness d1.

The luminous scale 30 may be formed by bonding a ring formed of hard rubber such as transparent silicone rubber to the cylindrical body 2, or may be molded integrally with the cylindrical body 2 using the same resin as that of the cylindrical body 2.

Furthermore, as the luminous scale, a band-like roughened surface may be formed on the outer surface of the cylindrical body in a peripheral direction, or a light diffusing agent-containing layer may be provided. The luminous scale roughened surface can be formed by subjecting it to blasting process or the like in the same manner as that for the roughened surface 20p at the distal end described above, and the light diffusing agent-containing layer can be formed in the same manner as that for the light diffusing agent-containing layer 20q at the distal end described above.

These configurations (the annular convex portion 30, the roughened surface, and the light diffusing agent-containing layer) may be appropriately combined. For example, the surface of the annular convex portion 30 may be subjected to the blasting process to form the luminous scale, or the annular convex portion 30 formed on the light diffusing agent-containing layer may be used as the luminous scale.

By providing the luminous scale 30 on the cylindrical body 2, as shown in examples described below, when the light that has been caused to enter the proximal end of the cylindrical body 2 is projected from the light-projecting portion 20 at the distal end, the light can also be emitted from the luminous scale 30 in the radially outward direction of the cylindrical body, and the resulting light-projecting portion can be observed as a scale mark-like pattern from the abdominal cavity side. Thus, the light-emitting portion by the luminous scale 30 can be used as a scale marker when the wall of the vagina or rectum is treated from the serosal membrane side at a desired position from the light-projecting portion (annular convex portion) 20 at the distal end.

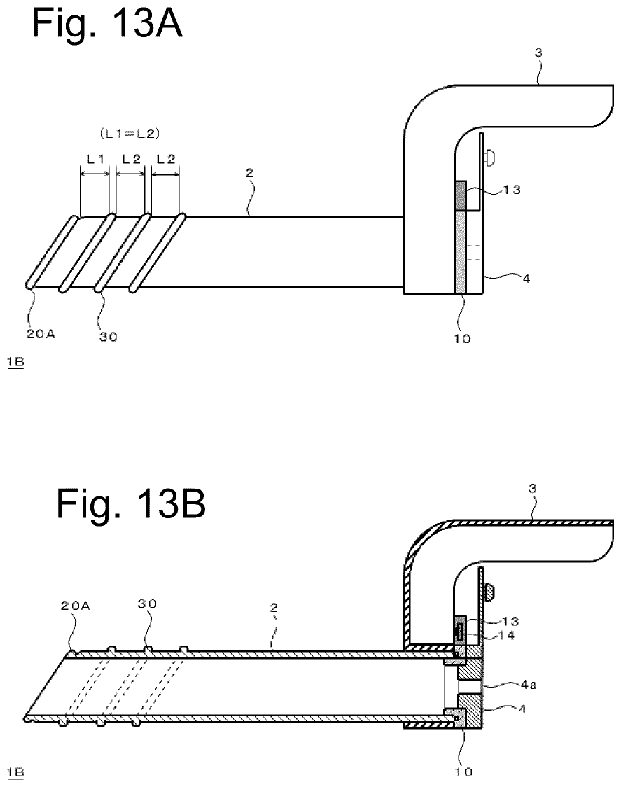

An insertion tool 1B illustrated in FIG. 13A and FIG. 13B is the insertion tool 1A illustrated in FIG. 1A, FIG. 1B, FIG. 1C, and FIG. 1D, in which the luminous scales (annular convex portions) 30 are provided around the entire periphery at an equal distance from the light-projecting portion (annular convex portion) 20A at the distal end. Thus, the surface surrounded by each luminous scale (annular convex portion) 30 is inclined with respect to the axis A of the cylindrical body 2, similar to the opening surface 2a of the cylindrical body 2. Note that, in the insertion tool 1B, the light-projecting portion (annular convex portion) 20 at the distal end and the luminous scales (annular convex portions) 30 are integrally formed with the cylindrical body 2.

By providing the luminous scales 30 around the entire periphery at an equal distance from the annular convex portion 20 at the distal end in this manner, it becomes easier to determine the distance from the vaginal fornix.

In the insertion tool of the present invention, the above-described various modifications can be combined as appropriate.

### EXAMPLE

### Emission Test 1

The insertion tool 1A illustrated in FIG. 1A, FIG. 1B, FIG. 1C, and FIG. 1D was produced using a Vagi pipe (M size) (pipe outer diameter 35 mm, inner diameter 29 mm, pipe effective length 180 mm) manufactured by Hakko Co., Ltd.

Specifically, eight surface-mounted chip LEDs (diameter 3 mm, emission wavelength 850 nm (near infrared) or 624 nm (red)) were arranged in a ring shape on the end face of the Vagi pipe on the handle side thereof (i.e., the proximal end of the cylindrical body 2) and fixed with an acrylic adhesive. Each chip LED 11 was connected to a battery 14 in a battery box 13. Furthermore, the annular convex portion 20 of the cylindrical body 2 was formed to have the cross section illustrated in FIG. 2.

The insertion tool 1A thus produced was covered with a 4 mm thick, light orange, silicone resin skin sheet, and each chip LED 11 was turned on. The skin sheet was photographed diagonally above the cylindrical body in a dark room with a near-infrared observation camera.

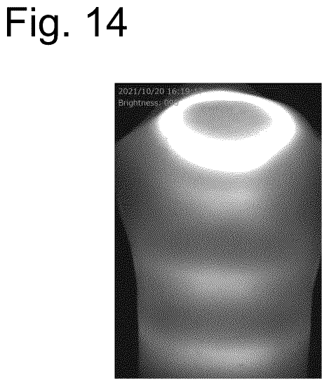

As a result, in both cases where the LED emission wavelength was 850 nm and 624 nm, the light emission from the light-projecting portion (annular convex portion) 20 at the distal end of the cylindrical body 2 and the light emission from the luminous scale (annular convex portion) 30 could be clearly confirmed on the skin sheet. The image in the case of 850 nm is illustrated in FIG. 14.

### Emission Test 2

An insertion tool (with tip R processing) was produced based on the insertion tool of the emission test 1, except that the luminous scale (annular convex portion) 30 was not provided, and the light-projecting portion at the distal end of the cylindrical body 2 was processed into the cross section illustrated in FIG. 2. Furthermore, an insertion tool (without tip R processing) was produced in the same manner, except that the distal end of the cylindrical body 2 was not subjected to the tip R processing, and the cross section of the distal end was formed as illustrated in FIG. 11.

The insertion tools produced with or without the tip R processing were each covered with a skin sheet in the Same manner as in the emission test 1, the LEDs were turned on, and the skin sheet was photographed in a dark room with the near-infrared observation camera.

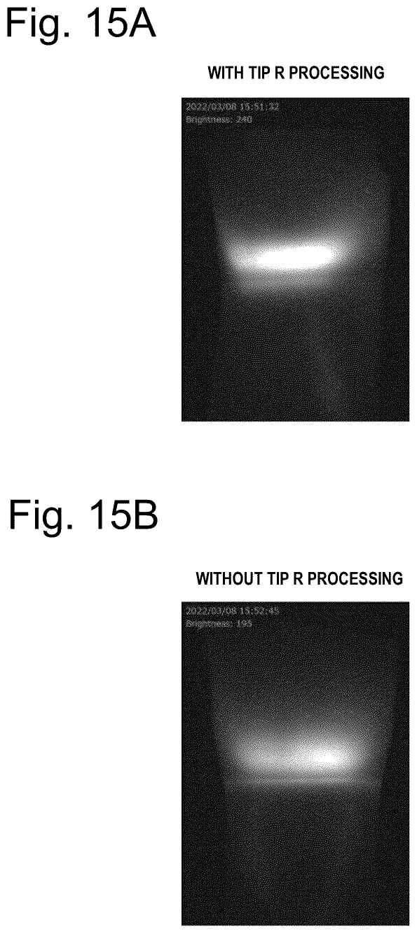

The results are illustrated in FIG. 15A (with tip R processing) and FIG. 15B (without tip R processing).

In FIG. 15A, the luminance (256 gradations) of the light-projecting portion exceeded 240, whereas the luminance of the light-projecting portion in FIG. 15B was 195. Because the luminance exceeding 240 was over-range for the camera used to take the photograph, the actual luminance intensity ratio is estimated to be 240/195 or more.

From these results, it was confirmed that the light intensity observed through the skin sheet was increased by the tip R processing.

### REFERENCE SIGNS LIST

1A, 1B insertion tool with light source
2 cylindrical body
2a opening surface of cylindrical body
2b inner surface of cylindrical body
2c outer surface of cylindrical body
2r region extending from corner at distal end toward proximal end
2x distal end of cylindrical body
2y inclined surface
3 handle
4 cap
4a opening
4b plug
10 light source, ring-shaped lighting device
11 chip LED
12 ring-shaped member
13 battery box
14 battery
20A, 20B, 20C, 20D light-projecting portion (annular convex portion)
20p light-projecting portion (roughened surface)
20q light-projecting portion (light diffusing agent-containing layer)
21 curved surface
22 reflective film
30 luminous scale, annular convex portion for luminous scale
100 vagina
101 vaginal fornix
110 uterus
120 tissue inside body cavity
200 endoscope
A axis of cylindrical body
B light-projecting portion
L1 distance between annular convex portion and annular convex portion for luminous scale
L2 interval between annular convex portions for luminous scale
L3 protruding length of annular convex portion for luminous scale
L4 width of annular convex portion for luminous scale in axis A direction
P1 inner end point of curved surface
P2 outer end point of curved surface
P3 outer protruding point of curved surface
X dissection line
d1 wall thickness of cylindrical body
d2 thickness of annular convex portion
d3 difference between thickness d2 of annular convex portion and wall thickness d1 of cylindrical body

1-11. (canceled)

12. An insertion tool, which is inserted into a natural opening of the body, comprising:
a light source which emits light without the use of an optical fiber at a proximal end of a cylindrical body formed of a light-conducting resin; and
a light-projecting portion at a distal end, wherein
the light-projecting portion has a single annular convex portion which protrudes with a curved surface that is convex in a radially outward direction of the cylindrical body, as a structure configured to project light, which has entered the proximal end, in the radially outward direction of the cylindrical body at the distal end,
at least the region of the curved surface including a protruding point in the radially outward direction in the center part of the curved surface comes into close contact with a tissue inside a body cavity when the insertion tool is inserted into the natural opening of the body, and
in a vertical section of the cylindrical body, the thickness of the curved surface at the protruding point is smaller than the wall thickness of the cylindrical body on the proximal end side from the annular convex portion.

13. The insertion tool according to claim 12, wherein an inner end point of an arc of the curved surface is on an inner surface of the cylindrical body in the vertical section of the cylindrical body.

14. The insertion tool according to claim 12, wherein the tip portion of the inner surface of the cylindrical body is inclined so that the opening diameter of the inner surface of the cylindrical body becomes wider as it approaches the tip of the cylindrical body.

15. The insertion tool according to claim 12, wherein an outer surface of the cylindrical body is a roughened surface in the light-projecting portion at the distal end.

16. An insertion tool, which is inserted into a natural opening of the body, comprising:
a light source which emits light without the use of an optical fiber at a proximal end of a cylindrical body formed of a light-conducting resin; and
a light-projecting portion at a distal end, wherein
the light-projecting portion has a single annular convex portion which protrudes with a curved surface that is convex in a radially outward direction of the cylindrical body, as a structure configured to project light, which has entered the proximal end, in the radially outward direction of the cylindrical body at the distal end,
at least the region of the curved surface including a protruding point in the radially outward direction comes into close contact with a tissue inside a body cavity when the insertion tool is inserted into the natural opening of the body, and
the light-projecting portion at the distal end includes a light diffusing agent.

17. An insertion tool, which is inserted into a natural opening of the body, comprising:
a light source which emits light without the use of an optical fiber at a proximal end of a cylindrical body formed of a light-conducting resin; and
a light-projecting portion at a distal end, wherein
the light-projecting portion has a single annular convex portion which protrudes with a curved surface that is convex in a radially outward direction of the cylindrical body, as a structure configured to project light, which has entered the proximal end, in the radially outward direction of the cylindrical body at the distal end,
at least the region of the curved surface including a protruding point in the radially outward direction comes into close contact with a tissue inside a body cavity when the insertion tool is inserted into the natural opening of the body, and
the cylindrical body includes a luminous scale on an outer peripheral surface at a predetermined distance from the light-projecting portion at the distal end, the luminous scale configured to emit light, which has entered the proximal end, in a scale mark-like pattern.

18. The insertion tool according to claim 17, wherein the luminous scale has the annular convex portion formed on an outer surface of the cylindrical body in a peripheral direction.

19. The insertion tool according to claim 17, wherein the luminous scale has a band-like roughened surface formed on the outer surface of the cylindrical body in the peripheral direction.

20. The insertion tool according to claim 17, wherein the luminous scale has a band-like light diffusing agent-containing layer formed on the outer surface of the cylindrical body in the peripheral direction.

21. The insertion tool according to claim 12, wherein the light source is a chip LED, a light-emitting surface of the chip LED is in contact with the proximal end of the cylindrical body, and a battery box is provided at the proximal end.

22. An insertion tool, which is inserted into a natural opening of the body, comprising:
a light source which emits light without the use of an optical fiber at a proximal end of a cylindrical body formed of a light-conducting resin; and
a light-projecting portion at a distal end of which the corners are rounded, wherein
the cylindrical body has a length such that the proximal end is located outside the body, when the distal end is inserted into the natural opening of the body and the light projected from the light-projecting portion to a tissue inside the body cavity is observed from the abdominal cavity side, and
the light-projecting portion is spaced apart from the proximal end, includes a light diffusing agent on an outer peripheral surface of the cylindrical body, and projects light, which has entered the proximal end, in a radially outward direction of the cylindrical body at the distal end.

23. An insertion tool, which is inserted into a natural opening of the body, comprising:
a light source which emits light without the use of an optical fiber at a proximal end of a cylindrical body formed of a light-conducting resin; and
a light-projecting portion at a distal end of which the corners are rounded, wherein
the light-projecting portion has a structure configured to project light, which has entered the proximal end, in a radially outward direction of the cylindrical body at the distal end, and
the cylindrical body includes a luminous scale on an outer peripheral surface at a predetermined distance from the light-projecting portion at the distal end, the luminous scale configured to emit light, which has entered the proximal end, in a scale mark-like pattern.

24. An insertion tool, which is inserted into a natural opening of the body, comprising:
a light source which emits light without the use of an optical fiber at a proximal end of a cylindrical body formed of a light-conducting resin; and
a light-projecting portion at a distal end of the cylindrical body, spaced apart from the proximal end, wherein
the corners of the distal end are rounded, the distal end comes into close contact with a tissue inside the body cavity when the insertion tool is inserted into the natural opening of the body, and
the light-projecting portion is a roughened surface formed by blasting process on the rounded corners and an outer peripheral surface adjacent to the corners at the distal end, and projects light, which enters the proximal end, in a radially outward direction of the cylindrical body on the roughened surface at the distal end.

25. The insertion tool according to claim 18, wherein the luminous scale has a band-like roughened surface formed on the outer surface of the cylindrical body in the peripheral direction.

26. The insertion tool according to claim 18, wherein the luminous scale has a band-like light diffusing agent-containing layer formed on the outer surface of the cylindrical body in the peripheral direction.

27. The insertion tool according to claim 16, wherein the light source is a chip LED, a light-emitting surface of the chip LED is in contact with the proximal end of the cylindrical body, and a battery box is provided at the proximal end.

28. The insertion tool according to claim 17, wherein the light source is a chip LED, a light-emitting surface of the chip LED is in contact with the proximal end of the cylindrical body, and a battery box is provided at the proximal end.
